(19) United States
(12) Patent Application Publication
(10) Pub. No.: US 2014/0212488 A1
(43) Pub. Date: Jul. 31, 2014

(54) ORAL TABLET FORMULATION CONSISTING OF IMMEDIATE RELEASE ROSUVASTATIN AND EXTENDED RELEASE METFORMIN

(71) Applicant: Althera Life Sciences LLC, Morristown, NJ (US)

(72) Inventor: Marie Charmaine Dias, Morristown, NJ (US)

(73) Assignee: Althera Life Sciences LLC, Morristown, NJ (US)

(21) Appl. No.: 14/250,610

(22) Filed: Apr. 11, 2014

Related U.S. Application Data
(63) Continuation-in-part of application No. PCT/US2013/031986, filed on Mar. 15, 2013.
(60) Provisional application No. 61/640,971, filed on May 1, 2012.

(51) Int. Cl. A61K 31/505; A61K 9/20; A61K 9/28; A61K 31/155

(52) U.S. Cl. CPC A61K 31/505; A61K 31/155; A61K 9/2095; A61K 9/2059; A61K 9/2054; A61K 9/2009; A61K 9/2031; A61K 9/2013; A61K 9/2086; A61K 9/28; A61K 9/2893
USPC 424/465; 514/275; 424/468; 264/129

(57) ABSTRACT

A solid dosage product and formulation containing a fixed combination of rosuvastatin and metformin is provided here. In the formulation rosuvastatin is delivered immediately to the patient and metformin is delivered in a slower controlled fashion over a longer course of time. A method to make the formulations as well as a method to treat patients with fixed combination solid dosage form of immediate release rosuvastatin and extended release metformin are provided here.

### PRIORITY

This application is Continuation in Part Application of international application number PCT/US2013/031986 filed on Mar. 15, 2013 and claiming priority of U.S. Provisional Application No. 61/612,563, filed on Mar. 19, 2012 and U.S. Provisional Application No. 61/640,971, filed on May 1, 2012. The contents of each of these applications are incorporated herein by reference in their entirety.

### BACKGROUND OF THE INVENTION

1. Field of invention

The present invention is directed to solid dosage product and formulation containing a fixed combination of rosuvastatin and metformin such that rosuvastatin is delivered immediately to the patient and metformin is delivered in a controlled fashion over a longer course of time, also described as extended release, as well as to methods of making such solid dosage forms and method of treating patients with fixed combination solid dosage forms of immediate release rosuvastatin and extended release metformin.

2. Related Background Art

Large scale clinical studies, epidemiological studies, and population reviews conducted by various organizations such as International Diabetes Federation (IDF), European Association for the Study of Diabetes (EASD), World Health Organization (WHO), National Cholesterol Education Program (NCEP), and Center for Disease Control (CDC) have identified Diabetes as a major risk factor for cardiovascular events and related mortality. The mortality follow-up of the EUROASPIRE I cohort showed that, apart from smoking, diabetes is the most important, single risk factor for coronary artery disease and subsequent cardiovascular morbidity and mortality. In addition, cardiovascular event is the main cause of death in patients with diabetes.

A number of clinical studies have shown that a substantial proportion of patients with diabetes do not reach recommended lipid targets with regard to Low Density Lipoproteins (LDL-C), High Density Lipoprotein (HDL-C), or Apolipoprotein (ApoB) as recommended by American Diabetes Association (ADA), The National Cholesterol Education Program (NCEP) and The European joint Task force of European Society of Cardiology (ESC) and the European Association for the Study of Diabetes (EASD), thereby keeping these patients at a high risk for premature cardiovascular disease and death.

There is significant existing evidence that fixed dose combination products improve adherence to medication and patient compliance due to reduced pill burden and improved ease of administration. It is been demonstrated that the adherence to medication in cardiovascular disease and in particular hyperlipidemia is less than desirable, which often results in an inability to meet treatment goals as recommended by European Society of Cardiology (ESC) and National Cholesterol Education Program (NCEP) ATP III. Adherence to anti-diabetes medication is an equally large problem as patients with type 2 diabetes often need 2-4 different anti-diabetic drugs to bring their HbAlc levels down to recommended levels. To make the pill burden even worse, many patients with diabetes and hyperlipedemia also suffer from hypertension as many of these patients have at least some degree of metabolic syndrome. Hence, any approach that can reduce the pill burden and ease adherence to medication could be very beneficial. The development of this invention needs to be looked at within this context. Patient adherence to medication has been shown to be significantly greater with a single-pill regimen compared with a two-pill regimen, or a two-pill regimen compared to a three- or four-pill regimen. Concerns about increasing a patient's pill burden often result in reluctance from physicians in adding further medications to a patient's existing regimen despite potential therapeutic benefits.

Accordingly, a fixed combination solid dosage formulation of metformin and rosuvastatin that is bioequivalent to corresponding free-combination would be highly desirable for the benefit of patients.

### SUMMARY OF THE INVENTION

In a first aspect, the present invention is an orally consumed fixed combination product that comprises immediate release rosuvastatin and extended release metformin, that is, metformin delivered to patient over an extended period of time that is indicated for simultaneous treatment of type 2 diabetes and hyperlipidemia or reduction in cardiovascular risk. This novel product delivers simultaneous treatment of diabetes and hyperlipidemia overcoming a significant dosing difference between the two individual ingredients and delivers a novel product that reduces low density cholesterol (LDL), potentially cardiovascular risk, and glucose levels in diabetes patients by simultaneously treating hyperglycemia and hyperlipidemia.

In a second aspect, the present invention is a novel formulation that comprises immediate release rosuvastatin and extended release metformin such that both rosuvastatin and extended release metformin in one tablet have same Area Under Curve as two active ingredients taken together individually orally, and pharmaceutically acceptable additives suitable for the preparation. In preferred embodiments of this invention, the rosuvastatin is in the form of rosuvastatin calcium and the pharmaceutically acceptable additives are selected from the diluents, disintegrants, glidants, lubricants, colorants and combinations thereof. The present invention overcomes the significant formulation challenge of timing: two different release timings of two different drugs.

In the preferred embodiments of this invention, the solid dosage form is a bi-layer tablet. The amount of rosuvastatin employed in such bi-layer tablets preferably ranges from about 5 mg to 40 mg, including 10 mg and 20 mg. The amount of metformin ranges from about 250 mg to 2000 mg, including 500 mg, 750 mg, 850 mg, 1000 mg, 1250 mg and 1500 mg.

In a third aspect, the present invention is directed to a method of making a solid dosage bi-layer form of rosuvastatin to be released in immediate form and metformin to be released in extended release. First stage is to develop the metformin extended release layer with the following steps: (a) Mix the intragranular ingredients in granulator, and granulate the above blend with binding agent; (b) Dry the granules partially, pass through mill and dry the sized granules until optimum LOD obtained; (c) Pass the dried granules through mill and sift the sized granules, and blend the granules; and (d) Lubricate the above granules with magnesium stearate. In the second stage, a rosuvastatin layer blend is created by blending the excipients with rosuvastatin in a blender, and lubricating the ingredients with lubricants such as magnesium stearate. In the third stage the above blends are compressed in bi-layer tablets with carefully controlled compression pressure. The compression pressure preferably being between 20 and 100 kNewtons and most preferably being between 40 and 80 kNewtons. Finally the bi-layer tablets are film-coated.

In a fourth aspect, this invention is directed to solid dosage forms of rosuvastatin and metformin made according to the method of the third aspect.

The fifth aspect of this invention is directed to a method of treating hyperlipidemia, cardiovascular diseases, simultaneously with type 2 diabetes comprising administering a solid dosage form of rosuvastatin and metformin to a patient in need of such a treatment. In a preferred embodiment, the solid dosage form is orally administered to the subject.

Accordingly it is an object of this invention to provide a solid dosage form of metformin and rosuvastatin combined in one tablet wherein rosuvastatin dosage is from about 5 mg to about 40 mg, and metformin dosage is from about 250 mg to about 2000 mg, and wherein rosuvastatin is released immediately and metformin is provided in granules releasing metformin in a controlled fashion.

It is another object of this invention to provide a method of treating hyperlipidemia, simultaneously with type 2 diabetes, said method comprising administering orally a solid dosage form of rosuvastatin and metformin to a patient in need of such a treatment, wherein the solid dosage form is one tablet comprising about 5 mg to 40 mg of rosuvastatin in immediately releasing form and about 250 mg to 2000 mg of metformin in delayed releasing form.

It is yet another object of this invention to provide a method to manufacture a solid dosage form of metformin and rosuvastatin combined in one tablet having an outer rosuvastatin layer with rosuvastatin dosage of about 5 mg to 40 mg and an inner metformin layer with metformin dosage of about 250 mg to 2000 mg, and wherein rosuvastatin is released immediately and metformin is provided in granules releasing metformin in a controlled fashion, said method comprising the steps of: a) Developing a blend for the metformin extended release layer with the steps of: i) Mixing intragranular ingredients in a granulator; and granulating resulting blend with a binding agent; ii) Drying the granules partially, sizing the granules by passing through a mill, and drying the sized granules until optimum LOD is obtained; iii) Sizing the dried granules by passing them through a mill, sifting the sized dried granules, and blending the resulting granules; and iv)Lubricating the resulting granules of step iii); b)Developing a blend for the rosuvastatin layer by blending selected excipients with rosuvastatin in a blender, and lubricating the blend with a lubricant;c) Compressing the blends of steps a) and b) into bi-layer tablets with a compression pressure ranging between 20 kilonewtons to 100 kilonewtons; and d) Providing a film-coat on the bi-layer tablets.

### BRIEF DESCRIPTION OF THE DRAWINGS

FIG. 1 : Average Release pattern of Concentration of Metformin and Rosuvastatin when administered to 10 healthy human volunteers, X-Axis depicts time, and Y-axis depicts % concentration of Metformin or rosuvastatin absorbed relative to maximum concentration over varying time points from 0 to 96 hours.

FIG. 2: Comparison of release pattern and concentration of Metformin in 10 healthy human volunteers between INVENTION and GLUCOPHAGE.

FIG. 3: Comparison of release pattern and concentration or Rosuvastatin in 10 healthy human volunteers between INVENTION AND CRESTOR.

### DETAILED DESCRIPTION OF THE INVENTION

The present invention is directed to solid dosage product and formulation containing a fixed combination of rosuvastatin and metformin such that rosuvastatin is delivered immediately to the patient and metformin is delivered in a controlled fashion over a longer course of time, also described as extended release, as well as to methods of making such solid dosage forms and method of treating patients with fixed combination solid dosage forms of immediate release rosuvastatin and extended release metformin.

The first embodiment of the invention is a fixed combination product that delivers metformin in an extended form and rosuvastatin in an immediate form to the patient. A number of clinical studies have shown that a substantial proportion of patients with diabetes do not reach recommended lipid targets with regard to Low Density Lipoproteins (LDL-C), High Density Lipoprotein (HDL-C), or Apolipoprotein (ApoB) as recommended by American Diabetes Association (ADA), The National Cholesterol Education Program (NCEP) and The European joint Task force of European Society of Cardiology (ESC) and the European Association for the Study of Diabetes (EASD), thereby keeping these patients at a high risk for premature cardiovascular disease and death. There is significant existing evidence that fixed dose combination products improve adherence to medication and patient compliance due to reduced pill burden and improved ease of administration. It is been demonstrated that the adherence to medication in cardiovascular disease and in particular hyperlipidemia is less than desirable, which often results in an inability to meet treatment goals as recommended by European Society of Cardiology (ESC) and National Cholesterol Education Program (NCEP) ATP III. Adherence to anti-diabetes medication is an equally large problem as patients with type 2 diabetes often need 2-4 different anti-diabetic drugs to bring their HbAlc levels down to recommended levels. To make the pill burden even worse, many patients with diabetes and hyperlipedemia also suffer from hypertension as many of these patients have at least some degree of metabolic syndrome. Hence, any approach that can reduce the pill burden and ease adherence to medication could be very beneficial. The development of this invention needs to be looked at within this context. Patient adherence to medication has been shown to be significantly greater with a single-pill regimen compared with a two-pill regimen, or a two-pill regimen compared to a three- or four-pill regimen. Concerns about increasing a patient's pill burden often result in reluctance from physicians in adding further medications to a patient's existing regimen despite potential therapeutic benefits.

Metformin and rosuvastatin are respectively well known treatments for diabetes and hyperlipidemia respectively. However, there is a significant challenge in combining the two products in one treatment. Rosuvastatin is indicated to be consumed or dosed once daily. However metformin is indicated for consumption at multiple points of time ranging as every 6-8 hours during the day. Given the difference in dosing pattern, this invention created a novel product where the product delivers rosuvastatin immediately when consumed by the patient, whereas metformin is released in a controlled fashion over a longer period of time thereby meeting the multiple dosing requirements of metformin. This invention hence creates a novel product that treats both diabetes and hyperlipidemia with a single oral dosage to be consumed by patients once daily.

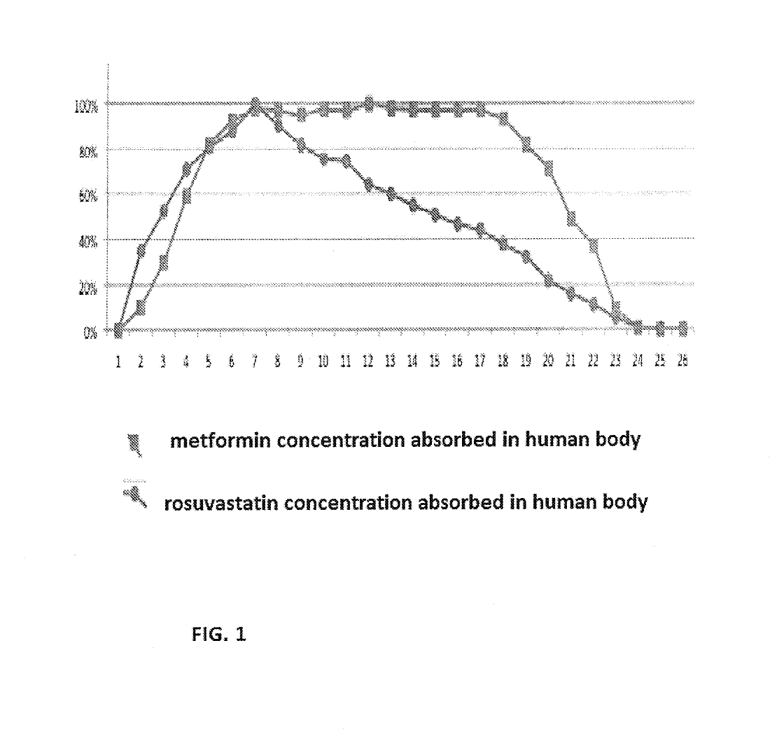

The first embodiment of this invention is described in the release pattern of the two components of the product as depicted in FIG. 1.

The second embodiment of this invention is a novel formulation that comprises of immediate release rosuvastatin and extended release metformin such that both rosuvastatin and extended release metformin in one tablet are expected to have same

Area Under Curve as two active ingredients taken together individually orally, and pharmaceutically acceptable additives suitable for the preparation. In preferred embodiments of this invention, the rosuvastatin is in the form of rosuvastatin calcium and the pharmaceutically acceptable additives are selected from the diluents, disintegrants, glidants, lubricants, colorants and combinations thereof. The present invention overcomes the significant formulation challenge of timing two different release timings of two different drugs.

In the preferred embodiments of this invention, the solid dosage form is a bi-layer tablet. The amount of rosuvastatin employed in such bi-layer tablets preferably ranges from about 5 mg to about 40 mg, including 10 mg and 20 mg. The amount of metformin ranges from about 250 mg to about 2000 mg, including 500 mg, 750 mg, 850 mg,1000 mg, 1250 mg and 1500 mg.

Creation of one tablet that has two different time-release of rosuvastatin and metformin is a significant challenge. Recognizing this challenge the inventors developed a strategy to use two layers—one of metformin and one of rosuvastatin. The strategy included rosuvastatin on the upper or outer layer of the tablet such that the release of rosuvastatin active ingredient is earlier than the metformin layer. And the metformin layer to include granules that control the release of metformin in the human stomach.

In this embodiment, the rosuvastatin layer of the formulation consists rosuvastatin as the active ingredient, with excipients such as starch 1-5% of the weight of the rosuvastatin layer, dicalcium phosphate 0.5-5% of the weight of the rosuvastatin layer, Cellulose 20-90% of the weight of the rosuvastatin layer, Crospovidone 2-30% of the weight of the rosuvastatin layer, and lubricants such as sodium stearyl fumarate 0.1-2% of the weight of the rosuvastatin layer. In this embodiment the metformin granules are created by combining metformin with cellulose 0.5-5% of the weight of the metformin layer, a polymer 5-50% of the weight of the metformin layer, and water and organically soluble cellulose such as hydroxypropyl cellulose 0.5-5% of the weight of the metformin layer. These granules are further packed in the polymer to the weight of 5-30% of the weight of the metformin layer along with a lubricant such as magnesium stearate 0.2-5% of the weight of the metformin layer.

In a third embodiment of the present invention is directed to a method of making a solid dosage bi-layer form of rosuvastatin to be released in immediate form and metformin to be released in extended release. First stage is to develop the metformin extended release layer with the following steps: (a) Mix the intragranular ingredients in granulator, and granulate the above blend with binding agent; (b) Dry the granules partially, pass through mill and dry the sized granules until optimum LOD obtained; (c) Pass the dried granules through mill and sift the sized granules, and blend the granules; and (d) Lubricate the above granules with magnesium stearate. In the second stage, a rosuvastatin layer blend is created by blending the excipients with rosuvastatin in a blender, and lubricating the ingredients with lubricants such as magnesium stearate. In the third stage the above blends are compressed in bi-layer tablets with carefully controlled compression pressure, preferably between 20 and 100 kNewton, and most preferably between 40 and 80 kNewton. Finally the bi-layer tablets are film-coated.

A key element of this embodiment of this invention is the compression pressure at which this particular bi-layer tablet is compressed to form the tablet. Given the desire for one ingredient of the tablet to be immediate release and another ingredient of the tablet to be extended release, at the same time they may be manufactured and compressed at the same time created significant challenges. For example, when the compression pressure was higher, the immediate release component of the rosuvastatin would release slower than was optimal, and if the compression pressure was too low, the extended release component of the metformin would release too fast. Example of this problem are described in below Table 1, which compares the release of a reference monotherapy tablet of rosuvastatin (stated as Crestor 20 mg) compared to a test tablet, stated as E11(T750/20).

TABLE 1
RELEASE OF ROSUVASTATIN IN THE TEST PRODUCT WITH
VARIABLE COMPRESSION COMPARED TO REFERENCE PRODUCT
(CRESTOR)
pH 6.6 Sodium Citrate Buffer, 900 mL, RPM-50, Type-II

| Time (min) | Crestor 20 mg | E111 (T750/20) |
|---|---|---|
| 0 | 0 | 0 |
| 10 | 91 | 77 |
| 20 | 95 | 83 |
| 30 | 97 | 85 |
| 45 | 99 | 87 |

The above problem was overcome by adjusting the compression pressure of the bi-layer machine to an optimum release pattern, which is an important part of the third embodiment of this invention, and ranges preferably between 20 and 500 KiloNewtons, more preferably between 20 and 100 Kilo Newtons and most preferably between 40 and 80 kiloNewtons.

Final embodiment of this invention is directed to a method of treating hyperlipidemia, cardiovascular diseases, simultaneously with type 2 diabetes comprising administering a solid dosage form of rosuvastatin and metformin to a patient in need of such a treatment. In a preferred embodiment, the solid dosage form is orally administered to the subject.

The invention is now described by means of non limiting examples.

### EXAMPLE 1

### Fixed Combination Tablet of Rosuvastatin 20 mg and Metformin 750 mg

| | Amount (mg) |
|---|---|
| Ingredients (metformin layer) | |
| Metformin HCl | 750.00 |
| Microcrystalline and Hydroxypropyl | 65.00 |
| Cellulose | |
| Polymer | 400.00 |
| Magnesium Stearate | 10.00 |
| Total | 1225.0 |
| Ingredients (rosuvastatin layer) | |
| Rosuvastatin calcium | 22.08 |
| Starch and microcrystalline cellulose | 292.58 |
| Crospovidone | 24.30 |
| Calcium hydrogen phosphate | 7.90 |
| dihydrate, Sodium Stearyl fumarate | |
| and Iron oxide red | |
| Total | 339.56 |

The above ingredients were used to manufacture a tablet in accordance with the third embodiment of this invention, where the steps undertaken were: First stage developed the metformin extended release layer with the following steps: (a) Mixed the intragranular ingredients in granulator, and granulated the above blend with binding agent; (b) Dried the granules partially, passed through mill and dried the sized granules until optimum LOD obtained; (c) Passed the dried granules through mill and sift the sized granules, and blended the granules; and (d) Lubricated the above granules with magnesium stearate. In the second stage, a rosuvastatin layer blend was created by blending the excipients with rosuvastatin in a blender, and lubricated the ingredients with lubricants such as magnesium stearate. In the third stage the above blends were compressed in bi-layer tablets with carefully controlled compression pressure. The pressure is preferably between 20 and 100 kiloNewtons, most preferably 40 and 80 kiloNewtons. Finally the bi-layer tablets were film-coated.

### EXAMPLE 2

### Fixed Combination Tablet of Rosuvastatin 10 mg and Metformin 500 mg

| | Amount (mg) |
|---|---|
| Ingredients (metformin layer) | |
| Metformin HCl | 500.00 |
| Microcrystalline and Hydroxypropyl | 43.33 |
| Cellulose | |
| Polymer | 266.67 |
| Magnesium Stearate | 6.67 |
| Total | 816.67 |
| Ingredients (rosuvastatin layer) | |
| Rosuvastatin calcium | 11.04 |
| Starch and microcrystalline cellulose | 146.29 |
| Crospovidone | 12.15 |
| Dicalcium phosphate, Sodium | 3.95 |
| Stearyl fumarate and ferric oxide red | |
| Total | 173.17 |

The above ingredients were used to manufacture a tablet in accordance with the third embodiment of this invention, where the steps undertaken were: First stage developed the metformin extended release layer with the following steps: (a) Mixed the intragranular ingredients in granulator, and granulated the above blend with binding agent; (b) Dried the granules partially, passed through mill and dried the sized granules until optimum LOD obtained; (c) Passed the dried granules through mill and sift the sized granules, and blended the granules; and (d) Lubricated the above granules with magnesium stearate. In the second stage, a rosuvastatin layer blend was created by blending the excipients with rosuvastatin in a blender, and lubricated the ingredients with lubricants such as magnesium stearate. In the third stage the above blends were compressed in bi-layer tablets with carefully controlled compression pressure. The pressure is preferably between 20 and 100 kiloNewtons, most preferably 40 and 80 kiloNewtons. Finally the bi-layer tablets were film-coated.

### Dissolution and Equivalence Results

Dissolution is a well-established method to test pharmacoequivalence of two products. The pharmacoequivalence of the fixed-combination dosage forms of the present invention was compared with that of the corresponding free-combinations.

Below are the results from the Example described earlier and of multiple tests that were undertaken between the test (fixed-combination) and the reference products (individual products) available in the market—GLUCOPHAGE SR and CRESTOR respectively.

TABLE 2
Dissolution profile of Metformin HCl in pit 4.5 media

| | 1000 mL | | |
|---|---|---|---|
| 4.5 Media | Reference- | 1000 RPM | |
| Time (hr) | GLUCOPHAGE SR | INVENTION | Specifications |
| 0 | 0 | 0 | 0 |
| 1 | 31 | 36 | 22-42% |
| 3 | 59 | 67 | 49-69% |
| 10 | 96 | 103 | NLT 85% |

NLT = Not Less Than

TABLE 3
Dissolution profile of Rosuvastatin Ca in pH 6.6 media

| | 900 mL | |
|---|---|---|
| USP-II | Reference- | 50 RPM |
| Time (minutes) | CRESTOR | INVENTION |
| 0 | 0 | 0 |
| 10 | 91 | 87 |
| 20 | 95 | 90 |
| 30 | 97 | 94 |
| 45 | 99 | 96 |

As is evident from the dissolution results, the invention is able to create a stable formulation that has similar drug release profiles for rosuvastatin and metformin such that metformin is released in an extended release fashion and rosuvastatin is released in an immediate release fashion.

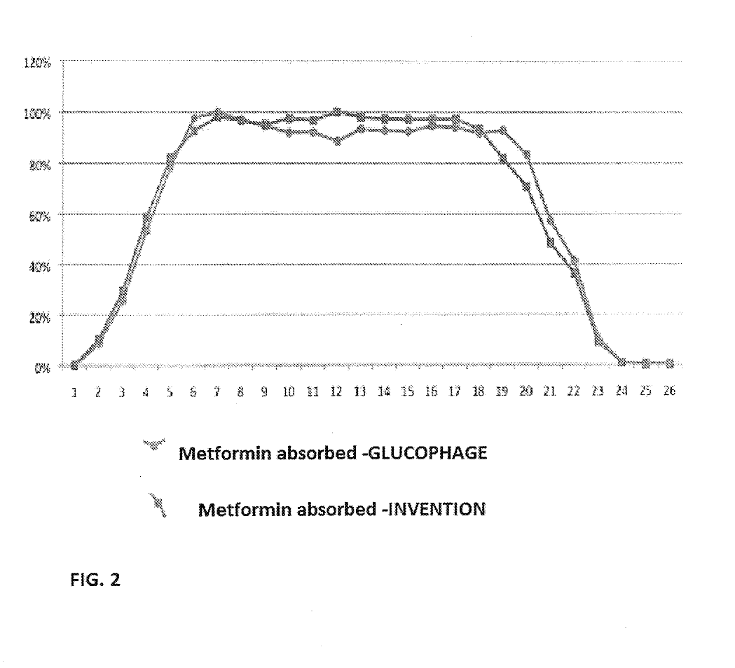

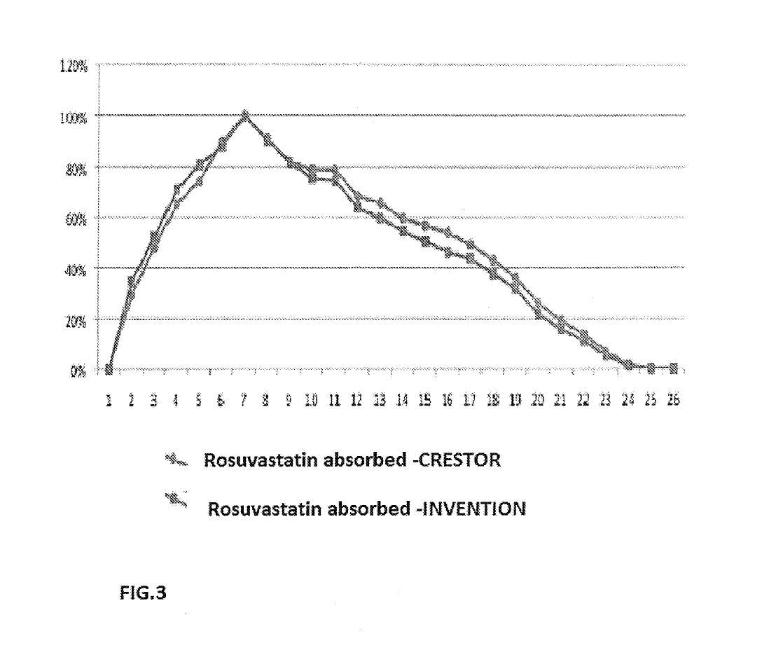

The individual components in the invention were also tested against the individual reference drugs, and were found to be of similar release pattern. These are provided in the FIG. 2 and FIG. 3.

While this invention has been particularly shown and described with references to preferred embodiments thereof, it will be understood by those skilled in the art that various changes in form and details may be made therein without departing from the scope of the invention encompassed by the appended claims.

1. A solid dosage form of metformin and rosuvastatin combined in one tablet wherein rosuvastatin dosage is from 5 mg to 40 mg, and metformin dosage is from 250 mg to 2000 mg, and wherein rosuvastatin is released immediately and metformin is provided in granules releasing metforminin a controlled fashion.

2. The solid dosage form of claim 1, wherein rosuvastatin and metformin are prepared in a bi-layer formulation.

3. The solid dosage form of claim 2, wherein the rosuvastatin and metformin are in a bi-layer tablet.

4. The solid dosage form in claim 2, wherein the rosuvastatin layer of the formulation comprises rosuvastatin as active ingredient, with excipients selected from the group consisting of starch 1-5% of the weight of the rosuvastatin layer, dicalcium phosphate 0.5-5% of the weight of the rosuvastatin layer, Cellulose 20-90% of the weight of the rosuvastatin layer, and crospovidone 2-30% of the rosuvastatin layer, and one or more lubricants.

5. The solid dosage form in claim 2, wherein metformin granules are created by combining metformin with cellulose 0.5-5% of the weight of the metformin layer, a polymer 5-20% of the weight of the metformin layer, water and organically soluble cellulose 0.5-5% of the weight of the metformin layer.

6. The solid dosage form of claim 6, wherein the metformin granules are further packed in the polymer to the weight of 5-30% of the weight of the metformin layer along with a lubricant 0.2-5% of the weight of the metformin layer.

7. The solid dosage form in claim 1 made by the process of manufacturing comprising the steps of :
a. developing a metformin extended release layer with the following steps:
i. Mixing intragranular ingredients in granulator, and granulating the blend with binding agent;
ii. Drying the granules partially, passing through mill and drying the sized granules until optimum LOD obtained;
iii. Passing the dried granules through mill and sifting the sized granules, and blending the granules;
iv. Lubricate the above granules with magnesium stearate.
b. developing a rosuvastatin layer blend by blending selected excipients with rosuvastatin in a blender, and lubricating the ingredients with lubricants
c. compressing the blends of step a) and b) in bi-layer tablets with compression pressure ranging between 20 and 100 kilonewtons ;and
d. wherein film-coating the bi-layer tablets.

8. The solid dosage form of claim 7, wherein the compression pressure in step c is between 40 and 80 kiloNewtons.

9. A method of treating hyperlipidemia, simultaneously with type 2 diabetes, said method comprising administering orally a solid dosage form of rosuvastatin and metformin to a patient in need of such a treatment, wherein the solid dosage form is one tablet comprising 5 mg to 40 mg of rosuvastatin in immediately releasing form and 250 mg to 2000 mg of metformin in delayed releasing form.

11. A solid dosage form of metformin and rosuvastatin combined in one tablet with 5 mg to 40 mg of rosuvastatin and 250 mg to 1000 mg of metformin,
wherein rosuvastatin is released immediately, and
metformin is released in controlled fashion and being provided in granules packed in a polymer, said granules comprising cellulose, a polymer, water and organically soluble cellulose.

12. The solid dosage form of claim 11, wherein metformin granules comprise 0.5-5% of the weight of the metformin layer, a polymer 5-50% of the weight of metformin layer, water and hydoxypropyl cellulose 0.5-5% of the weight of the metformin layer and 0.2-5% magnesium of the weight of the metformin layer.

13. The solid dosage form of claim 12, wherein the granules are packed in polymer 5-30% of the weight of the metformin layer.

14. The solid dosage form of metformin and rosuvastatin of claim 11, wherein one tabled contains 10-25 mg rosuvastatin and 500 -750 mg metformin.

15. The solid dosage form of claim 5, wherein the lubricant is sodium stearyl fumarate 0.1-2% of the weight of the rosuvastatin layer.

16. The solid dosage form of claim 6, wherein the organically soluble cellulose is hydroxypropyl cellulose.

17. The solid dosage form of claim 7, wherein the lubricant is magnesium stearate.

18. The solid dosage form of claim 8, wherein the lubricant in step b) is magnesium stearate.

19. A method to manufacture a solid dosage form of metformin and rosuvastatin combined in one tablet having an outer rosuvastatin layer with rosuvastatin dosage of 5 mg to 40 mg and an inner metformin layer with metformin dosage of 250 mg to 2000 mg, and wherein rosuvastatin is released immediately and metformin is provided in granules releasing metformin in a controlled fashion, said method comprising the steps of:
a) Developing a blend for the metformin extended release layer with the steps of:
i) Mixing intragranular ingredients in a granulator; and granulating resulting blend with a binding agent;
ii) Drying the granules partially, sizing the granules by passing through a mill, and drying the sized granules until optimum LOD is obtained;
iii) Sizing the dried granules by passing them through a mill, sifting the sized dried granules, and blending the resulting granules; and
iv) Lubricating the resulting granules of step iii);
b) Developing a blend for the rosuvastatin layer by blending selected excipients with rosuvastatin in a blender, and lubricating the blend with a lubricant;
c) Compressing the blends of steps a) and b) into bi-layer tablets with a compression pressure ranging between 20 kilonewtons to 100 kilonewtons; and
d) Providing a film-coat on the bi-layer tablets.

20. The method of claim 19, wherein rosuvastatin dosage in the outer layer is 10-25 mg, and metformin dosage in the inner layer is 500-750 mg, and the pressure in step c) is 40 to 80 kilonewtons.
